(19) United States
(12) Patent Application Publication
(10) Pub. No.: US 2006/0228375 A1
(43) Pub. Date: Oct. 12, 2006

(54) Induction of mucosal immunity by vaccination via the skin route

(75) Inventors: Dexiang Chen, Madison, WI (US); Sangeeta Bhargava, Madison, WI (US); Deborah Fuller, Madison, WI (US)

Correspondence Address: FOLEY AND LARDNER LLP;SUITE 500, 3000 K STREET NW, WASHINGTON, DC, 20007, US

(73) Assignee: PowderJect Vaccines, Inc.

(21) Appl. No.: 11/453,977

(22) Filed: Jun. 16, 2006

Related U.S. Application Data
(63) Continuation of application No. 10/724,727, filed on Dec. 2, 2003, now abandoned.
Application No. 10/724,727 is a continuation of application No. 09/710,104, filed on Nov. 9, 2000, now abandoned.
(60) Provisional application No. 60/164,529, filed on Nov. 10, 1999.

(51) Int. Cl. A61K 39/12; A61K 39/02; A61K 39/106; A61K 48/00

(52) U.S. Cl. 424/204.1; 424/234.1; 424/261.1; 514/44

(57) ABSTRACT

Methods for generating an immune response at a mucosal surface are described. Compositions suitable for use in the methods for generating an immune response at a mucosal surface are also described. In addition, methods for treating or preventing a disease caused by the entry of a pathogen into the body of a subject via a mucosal surface are provided.

### CROSS-REFERENCE TO RELATED PATENT APPLICATIONS

This application is a Continuation of U.S. application Ser. No. 10/724,727, filed Dec. 2, 2003, incorporated herein by reference in its entirety, which is a Continuation of U.S. application Ser. No. 09/710,104 (now abandoned), filed Nov. 9, 2000, incorporated herein by reference in its entirety, which claims priority top U.S. provisional application Ser. No. 60/164,529, filed Nov. 10, 1999, pursuant to 35 U.S.C. § 119(e)(1) and which is incorporated herein by reference in its entirety.

### BACKGROUND OF THE INVENTION

Mucosa is the portal entry for variety of pathogens such as bacteria, virus, and parasites. Therefore, vaccines or vaccination methods that induce protective immunity (both antibody and cellular immunity) at the mucosal surface are needed for adequate protection against many diseases such as AIDS, pneumococcal diseases, and influenza. It is commonly thought that vaccination by parenteral injection using a syringe and needle does not lead to a mucosal immune response and that mucosal immunity can only be generated by direct application of vaccines to the mucosal tissue. Topical application of non-replicating vaccines to the mucosal surface has not met with much success yet because of the low delivery efficiency.

Immunization via the skin route leads to serum antibody responses. Methods that deliver vaccines via the skin routes include intradermal injection using a liquid injector, transcutaneous immunization (TCI), patch deliver), and our recently described epidermal powder injection technology. Compared to other skin immunization methods, powder injection uses smaller vaccine doses and elicits higher serum antibody titers. Very importantly, a variety of adjuvants can be co-formulated and delivered with a vaccine to enhance and modulate the immune response with the powder injection method.

Skin is not a mucosal tissue; therefore, vaccination via the skin is not expected to cause a mucosal response. However, Glenn G. M. et al. have recently reported IgA antibody response to cholera toxin, but not to a co-administered diphtheria toxoid, after transcutaneous immunization (Glenn et al. (1998) J. Immunol. 161:3211-3214; Glenn et al. (1999) Infect. Immun. 67:1100-1106). In these studies, the authors stated that, as expected, no mucosal IgA antibodies to co-administered diphtheria toxoid were found. The dose of cholera toxin used in these studies was 100 mg. TCI is a technique that utilizes ADP-ribosylating exotoxins (e.g. cholera toxin etc) to deliver liquid vaccines through a non-perforated intact skin.

### SUMMARY OF THE INVENTION

The present invention provides a new method that elicits mucosal immunity to antigens that are preferably co-formulated with an adjuvant or adjuvant combination and delivered using a powder injection technique. This invention differs from TCI methodology in at least four aspects: 1) Powder injection delivers particulate vaccine compositions by perforating the stratum corneum, TCI delivers liquid vaccine compositions by coating them onto intact non-perforated skin; 2) Powder injection elicits mucosal immune responses to vaccine antigens, while TCI only elicits mucosal IgA to cholera toxin delivery vehicle/adjuvant, but not to co-administered antigen; 3) In addition to ADP-ribosylating exotoxins, other adjuvants such as CpG can be used to induce and enhance a mucosal response in the powder injection method, while TCI is dependent on ADP-ribosylating exotoxins for delivery; and 4) Powder injection uses smaller doses of vaccine and adjuvants than TCI. We have conducted studies to demonstrate that TCI could not elicit a detectable mucosal antibody response to either cholera toxin or co-administered vaccine with the adjuvant and vaccine doses that were used in the powder injection system. The invention also provides a vaccine composition for use in such a method. A method for treating a disease caused by a pathogen that enters the body via a mucosal surface is also provided.

Accordingly the invention provides a method of generating an immune response at a mucosal surface comprising delivering a particulate vaccine composition into or across the skin of a vertebrate subject using a transdermal delivery technique, wherein the vaccine composition comprises an antigen or a nucleic acid encoding an antigen. Preferably, the particulate vaccine composition is delivered using a needleless syringe powder injection device.

Further, an adjuvant composition may be delivered to the vertebrate subject. The adjuvant composition may be administered to the same or different site to the vaccine composition. The adjuvant composition may also be delivered prior to, subsequent to, or concurrently with the vaccine composition. The adjuvant and vaccine compositions may be administered in either a single composition or as separate compositions. In one particular embodiment, the adjuvant composition is in particulate form and delivered using a transdermal delivery technique, preferably using a needleless syringe powder injection device.

In a preferred embodiment the adjuvant composition comprises a combination of two or more adjuvants. In a particularly preferred embodiment the adjuvant composition comprises a cholera toxin and an oligonucleotide containing a CpG motif.

In another embodiment, the invention provides a particulate vaccine composition suitable for delivery into or across the skin of a vertebrate subject using a transdermal delivery technique, the said composition comprising an antigen, or a nucleic acid encoding said antigen, an ADP-ribosylating toxin as an adjuvant, and an oligonucleotide containing a CpG motif. In a preferred embodiment, the ADP-ribosylating toxin is a cholera toxin. These compositions find particular use as therapeutics or vaccines that can be administered to the skin yet provide mucosal immunity against a pathogen that enters the body via a mucosal surface. Preferably, the particulate vaccine composition is delivered using a needleless syringe powder injection device.

In yet another embodiment, the invention provides a method for treating or preventing a disease caused by the entry of a pathogen into the body of a vertebrate subject via a mucosal surface. The method entails delivering a particulate vaccine composition into or across the skin of a vertebrate subject in need of treatment or vaccination using a transdermal delivery technique, wherein the vaccine composition comprises an antigen or nucleic acid encoding an antigen, and then coadministering an adjuvant composition to the subject. The adjuvant composition comprises an ADP-ribosylating toxin and coadministration of the vaccine and adjuvant compositions is sufficient to bring about a mucosal immune response specific for the antigen. It is preferred that the ADP-ribosylating toxin is a cholera toxin.

These and other embodiments of the invention will readily occur to those of ordinary skill in the art in view of the disclosure herein.

### BRIEF DESCRIPTION OF THE DRAWINGS

FIGS. 1A and 1B illustrate the mucosal antibody response generated by a diphtheria toxoid vaccine composition delivered by a powder injection technique. Mice were vaccinated with two 5 μg doses of diphtheria toxoid formulated with 5 μg of cholera toxin on weeks 0 and 4. FIG. 1A shows the IgG titer and FIG. 1B shows the IgA titer in saliva determined by ELISA from individual mice at week 6.

FIG. 2 compares the serum antibody response induced by a diphtheria toxoid vaccine composition delivered either via a powder injection technique or by a TCI delivery technique. Mice were vaccinated with two 5 μg doses of diphtheria toxoid formulated with 5 μg of cholera toxin on weeks 0 and 4. The serum IgG titers determined by ELISA in pooled sera from 8 mice on week 6 are shown. Abbreviations DT: (diphtheria toxoid); CT: (cholera toxin); TCI: (transcutaneous immunization); PowderJect: (transdermal powder injection).

FIG. 3 compares the mucosal response to elicited by a diphtheria toxoid vaccine composition delivered either via a powder injection technique or by a TCI delivery technique. Mice were vaccinated with two 5 μg doses of diphtheria toxoid formulated with 5 μg of cholera toxin on weeks 0 and 4. Tissue fragments were collected on week 6 and cultured for 7 days in vitro. Tissue culture supernatant was assayed for antibodies to diphtheria toxoid in an ELISA. Mean IgG levels from 8 mice are shown. Abbreviations: PP: (Peyer's patch); MLN: (Mesenteric lymph node); CT: (cholera toxin); DT: (diphtheria toxoid); PJ: (transdermal powder injection); TCI: (transcutaneous immunization).

FIG. 4 compares the levels of IgG antibodies in nasal secretions produced in response to a diphtheria toxoid vaccine composition delivered via (1) a powder injection technique, (2) a TCI delivery technique or by intraperitoneal injection via conventional needle and syringe. Mice were vaccinated with two 5 μg doses of diphtheria toxoid formulated with 5 μg of cholera toxin on weeks 0 and 4. Nasal secretions were collected on week 6 and assayed for antibodies to diphtheria toxoid in an ELISA. IgG titers in four individual animals are shown. Abbreviations: IP: (intraperitoneal injection using a needle); TCI: (transcutaneous immunization); PowderJect: (transdermal powder injection).

FIG. 5 compares the levels of IgA antibodies to the influenza vaccine (present in the culture supernatant of trachea) produced following delivery of an influenza vaccine composition (either with or without a cholera toxin adjuvant) delivered via powder injection or TCI of influenza vaccine. Mice were vaccinated with two 5 μg doses of influenza vaccine on weeks 0 and 4. Trachea was collected on week 6 and cultured in vitro for 7 days. IgA antibodies in the culture supernatant were measured in an ELISA. IgA titers from 6 individual animals for each immunization schedule are shown. Abbreviations: Flu: (influenza vaccine); CT: (cholera toxin); PJ: (transdermal powder injection); TCI: (transcutaneous immunization).

FIG. 6 compares the levels of IgG antibodies to the influenza vaccine (in nasal secretions) produced following administration of an influenza vaccine composition (either with or without cholera toxin adjuvant) delivered via powder injection or TCI. Mice were vaccinated with two 5 μg doses of influenza vaccine on weeks 0 and 4. Nasal secretions were collected on week 6 for antibody assay in an ELISA. Mean IgG antibody titers from 6 animals are shown. Abbreviations: Flu: (influenza vaccine); CT: (cholera toxin); PJ: (transdermal powder injection); TCI: (transcutaneous immunization); SI: (small intestine); PP: (Peyer's patch); MLN: (Mesenteric lymph node).

FIG. 7 compares the levels of IgG antibodies to influenza vaccine (in the culture supernatant of mucosal tissues) produced following administration of an influenza vaccine composition (with or without cholera toxin adjuvant) delivered by powder injection or TCI. Mice were vaccinated with two 5 μg doses of influenza vaccine on weeks 0 and 4. Mucosal tissues were collected on week 6 and cultured in vitro for 7 days. IgG antibodies in the culture supernatant were measured in an ELISA. IgG titers from 6 individual animals are shown. Abbreviations: Flu: (influenza vaccine); CT: (cholera toxin); PJ: (transdermal powder injection); TCI: (transcutaneous immunization).

FIGS. 8A-8D compare the levels of IgA antibodies to influenza vaccine (in mucosal secretions) following powder injection of an influenza vaccine (a) without adjuvant, (b) with cholera toxin adjuvant, (c) with a CpG motif-containing oligonucleotide adjuvant, or (d) with a combination of the two adjuvants. Mice were vaccinated with two 5 μg doses of influenza vaccine on weeks 0 and 4. Mucosal secretions were collected on week 6 and assayed in an ELISA. FIG. 8A shows Mean IgA titers in nasal secretions from 8 animals. FIG. 8B shows Mean IgA titers in the saliva from 8 animals. FIG. 8C shows IgA titers in vaginal washes pooled from 8 animals. FIG. 8D shows Mean IgA titers in fecal extract from 8 animals. Abbreviations: Flu: (influenza vaccine); PR8: (influenza vaccine); CT: (cholera toxin); CpG: (oligonucleotide containing CpG motif); PJ: (transdermal powder injection).

FIG. 9 compares the levels of IgA antibodies to the influenza vaccine (from different in vitro cultured mucosal tissues) following powder injection of an influenza vaccine composition (a) without adjuvant, (b) with cholera toxin adjuvant, (c) with a CpG motif-containing oligonucleotide adjuvant, (d) or with a combination of the two adjuvants. Mice were vaccinated with two 5 μg doses of influenza vaccine on weeks 0 and 4. Mucosal tissues were collected on week 6, cultured in vitro for 7 days, and assayed in an ELISA. Mean IgA titers from 8 mice are shown. Abbreviations: PP: (Peyer's patch); SI: (small intestine); PR8: (the PR8 influenza vaccine); CT: (cholera toxin); CpG: (CpG motif-containing oligonucleotide); PJ: (transdermal powder injection).

FIGS. 10A-10C compare the levels of IgG antibodies to the influenza vaccine (in mucosal secretions and in vitro cultured mucosal tissues) following powder injection of an influenza vaccine composition (a) without adjuvant, (b) with cholera toxin adjuvant, (c) with a CpG motif-containing oligonucleotide adjuvant, or (d) with a combination of the two adjuvants. Mice were vaccinated with two 5 μg doses of influenza vaccine on weeks 0 and 4. Mucosal secretions were collected on week 6 and assayed in an ELISA. Mucosal tissues collected on week 6 were cultured in vitro for 7 days, and the culture supernatant was assayed for influenza-specific antibodies in an ELISA. FIG. 10A shows mean IgG titers in the culture supernatant of various mucosal tissues from 8 animals. FIG. 10B shows mean IgG titers in the fecal extract from 8 animals. FIG. 10C shows IgG titers in the saliva pooled from 8 animals. Abbreviations: PP: (Peyer's patch); SI: (small intestine); MLN: (Mesenteric lymph node); Flu: (influenza vaccine); CT: (cholera toxin); CpG: (oligonucleotide containing CpG motif); PJ: (transdermal powder injection).

FIG. 11 shows the levels of IgG antibodies to the influenza antigen following powder injection of a commercial influenza vaccine (a) without adjuvant, (b) with cholera toxin adjuvant, or (c) a combination of the cholera toxin adjuvant with a CpG motif-containing oligonucleotide adjuvant. 8 mice/group received the various vaccine compositions, and sera samples were collected 4 weeks after immunization. The data are IgG titer of the individual animals (∘) and the mean titer of the 8 mice in the group (−). Abbreviations: no adj.: (without adjuvant); CT: (cholera toxin); CpG: (oligonucleotide containing CpG motif).

FIG. 12 shows the protection against mortality in vaccinated mice following challenge with a heterologous influenza strain. Mice were vaccinated via powder injection of a commercial influenza vaccine (a) without adjuvant, (b) with cholera toxin adjuvant, or (c) a combination of the cholera toxin adjuvant with a CpG motif-containing oligonucleotide adjuvant. 8 mice/group received the various vaccine compositions, and challenged with 10× the LD₅₀ of a mouse-adapted influenza (Aichi/68 (H₃N₂)) virus administered intranasally. All animals receiving powder injection of vaccine composition with either the cholera toxin adjuvant alone (□), or the cholera toxin adjuvant with a CpG motif-containing oligonucleotide adjuvant (*) survived the entire 21 day monitoring period as contrasted with 0% of the non-vaccinated control animals (∘) surviving past 8 days post challenge, and only 40% of the animals vaccinated with vaccine composition alone (⋄) surviving past 10 days post challenge. Abbreviations: no adj. (without adjuvant); CT: (cholera toxin); CpG: (oligonucleotide containing CpG motif).

FIG. 13 shows influenza antigen-specific IgA secreting cells present in mucosal tissue samples obtained from mice vaccinated via powder injection of a commercial influenza vaccine adjuvanted with a combination of cholera toxin adjuvant and a CpG motif-containing oligonucleotide adjuvant. Control animals were vaccinated with the same adjuvanted influenza vaccine composition (in phosphate buffered saline) via either intranasal administration or subcutaneous injection with a conventional needle and syringe. 8 mice/group received a prime (day 0) and boost (day 28) immunization with the various vaccine compositions, and mucosal tissue samples were collected two weeks after the boost immunization. Cell suspensions were prepared from pooled samples, and influenza virus antigen-specific IgA secreting cells were enumerated using an ELISPOT assay. The data shown in the figure are average IgA spct-forming cell (SPC) frequency in pooled mucosal tissue from eight mice. Abbreviations: IN (intranasal); EI (transdermal powder injection); SC (subcutaneous); PP (Peyer's patch).

### DETAILED DESCRIPTION OF THE PREFERRED EMBODIMENTS

Before describing the present invention in detail, it is to be understood that this invention is not limited to particular pharmaceutical formulations or process parameters as such may, of course, vary. It is also to be understood that the terminology used herein is for the purpose of describing particular embodiments of the invention only, and is not intended to be limiting.

It must be noted that, as used in this specification and the appended claims, the singular forms “a,” “an” and “the” include plural referents unless the content clearly dictates otherwise. Thus, for example, reference to “a particle” includes two or more particles, reference to “an antigen” or “an adjuvant” includes a mixture or a combination of two or more such agents, reference to “an excipient” includes mixtures of two or more excipients, and the like.

### A. Definitions

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed, and are intended to be defined as indicated below.

As used herein, the term “transdermal delivery” includes intradermal (e.g., into the dermis or epidemis) and transdermal (e.g. “percutaneous”) i.e., delivery by passage of an agent into or through at least a top layer of skin. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Bemer (eds.), CRC Press, (1987). The term expressly encompasses delivery of a particulate agent to target tissue to provide a mucosal immune response, and particularly includes delivery of the agent via a powder injection technique.

By “needleless syringe” is meant an instrument which can be used to carry out a powder injection technique, and is used herein to deliver a particulate composition transdermally, without a conventional needle that pierces the skin. Suitable needleless syringes for use with the present invention are discussed throughout this document.

An “antigen” refers to any immunogeneic moiety or agent, generally a macromolecule, which can elicit an immunological response in an individual. The term may be used to refer to an individual macromolecule or to a homogeneous or heterogeneous population of antigenic macromolecules. As used herein, the term “antigen” includes allergens. Thus, antigens include proteins, polypeptides, antigenic protein fragments, oligosaccharides, polysaccharides, and the like. Furthermore, the antigen can be derived from any virus, bacterium, parasite, protozoan, or fungus, and can be a whole organism. Typically an antigen will be derived from a pathogen that enters the body via a mucosal surface. The term also includes tumor antigens derived from tumors of mucosal tissue (for example, tumors of the oesophagus, lung, stomach, cervix etc.). Similarly, an oligonucleotide or polynucleotide which expresses an antigen, such as in DNA immunization applications, is also included in the definition of antigen. Synthetic antigens are also included, for example, polyepitopes, flanking epitopes, and other recombinant or synthetically derived antigens (Bergmann et al. (1993) Eur. J. Immunol. 23:2777-2781; Bergmann et al. (1996). J. Immunol. 157:3242-3249; Suhrbier, A. (1997) Immunol. and Cell Biol. 75:402-408; Gardner et al. (1998) 12th World AIDS Conference, Geneva. Switzerland, Jun. 28-Jul. 3, 1998).

The term “vaccine composition” intends any pharmaceutical composition containing an antigen, which composition can be used to prevent or treat a disease or condition in a subject. The term thus encompasses both subunit vaccines, i.e., vaccine compositions containing antigens which are separate and discrete from a whole organism with which the antigen is associated in nature, as well as compositions containing whole killed, attenuated or inactivated pathogens including bacteria, viruses, parasites and other microbes. Vaccine compositions may also contain one or more adjuvants as described herein. Typically the vaccine composition is used for the prophylaxis of a disease caused by a pathogen that enters the body via a mucosal surface.

The term “mucosal surface” includes all external surfaces of the body which are protected by the secretion of mucus. Such mucosal surfaces include those of the gastrointestinal tract, the respiratory tract and the reproductive tract. Also included in this definition is the eye and urinary tract. The skin is not a mucosal surface.

An “immunological response” or “immune response” against a selected agent, antigen or a composition of interest is the development in an individual of a humoral and/or a cellular immune response to molecules (e.g., antigen) present in the agent or composition of interest. For purposes of the present invention, a “humoral immune response” refers to an immune response mediated by antibody molecules, while a “cellular immune response” is one mediated by T-lymphocytes and/or other white blood cells.

By “mucosal immune response” is meant the generation of immunity against an antigen or infectious agent (either in a humoral or a cellular context) at a mucosal surface. The generation of humoral immunity at the mucosal surface involves the secretion of antibodies into the mucus. Antibodies of the IgG subclass may be present in the mucus. However, the hallmark of a mucosal immune response is the generation of IgA antibodies. IgA is the major antibody subclass in mucosal secretions where it functions to block the entry of microorganisms from the external surfaces to the tissues below. By “antigen-specific IgA response” is meant the production of IgA anitibodies directed against a specific antigen. The generation of an antigen-specific immune response is an important part of the generation of a mucosal immune response.

The term “adjuvant” intends any material or composition capable of specifically or non-specifically altering, enhancing, directing, potentiating or initiating an antigen-specific immune response. Thus, coadministration of an adjuvant and an antigen (e.g., as a vaccine composition) may result in a lower dose or fewer doses of antigen being necessary to achieve a mucosal immune response in the subject to which the antigen is administered. The effectiveness of an adjuvant can be determined by administering the adjuvant with a vaccine composition and vaccine composition controls to animals and comparing antibody titers and/or cellular-mediated immunity against the two using standard assays such as radioimmunoassay, ELISAs, CTL assays, and the like, well known in the art. Typically, in a vaccine composition, the adjuvant is a separate moiety from the antigen, although a single molecule can have both adjuvant and antigen properties (e.g., cholera toxin). For the purposes of the present invention, an adjuvant is used to either enhance the immune response to a specific antigen, e.g., when an adjuvant is coadministered with a vaccine composition, the mucosal immune response is greater than the immune response elicited by an equivalent amount of the vaccine composition administered without the adjuvant, or the adjuvant is used to direct a mucosal immune response against an antigen administered to the skin. In addition, for the purposes of the present invention, an “effective amount” of an adjuvant will be that amount which enhances an immunological response to a coadministered antigen in a vaccine composition such that lower or fewer doses of the antigen are required to generate an efficient mucosal immune response.

An “adjuvant composition” intends any pharmaceutical composition containing an adjuvant. Adjuvant compositions can be delivered in the methods of the invention while in any suitable pharmaceutical form, for example, as a liquid, powder, cream, lotion, emulsion, gel or the like. However, preferred adjuvant compositions will be in particulate form.

Similarly, an “effective amount” of an antigen or vaccine composition is an amount which will stimulate an antigen-specific mucosal immune response in the subject to which the vaccine composition is administered. The immune response may be a humoral, cell-mediated and/or protective mucosal immune response it is preferred that the mucosal immune response is, or includes, a IgA response at a mucosal surface.

As used herein, the term “coadministered,” such as when an adjuvant is “coadministered” with an antigen (e.g., a vaccine composition), intends either the simultaneous or concurrent administration of adjuvant and antigen, e.g., when the two are present in the same composition or administered in separate compositions at nearly the same time but at different sites, as well as the delivery of adjuvant and antigen in separate compositions at different times. For example, the adjuvant composition may be delivered prior to or subsequent to delivery of the antigen at the same or a different site. The timing between adjuvant and antigen deliveries can range from about several minutes apart, to several hours apart, to several days apart.

Particles which comprise an antigen or adjuvant are typically prepared as pharmaceutical compositions which can contain one or more added materials such as carriers, vehicles, and/or excipients. “Carriers,” “vehicles” and “excipients” generally refer to substantially inert materials which are nontoxic and do not interact with other components of the composition in a deleterious manner. These materials can be used to increase the amount of solids in particulate compositions. Examples of suitable carriers include water, silicone, gelatin, waxes, and like materials. Examples of normally employed “excipients” include pharmaceutical grades of dextrose, sucrose, lactose, trehalose, mannitol, sorbitol, inositol, dextran, starch, cellulose, sodium or calcium phosphates, calcium carboonate, calcium sulfate, sodium citrate, citric acid, tartaric acid, glycine, high molecular weight polyethylene glycols (PEG), and combinations thereof.

As used herein, the term “treatment” includes any of following the prevention of infection or reinfection the reduction or elimination of symptoms; and the reduction or complete elimination of a pathogen. Treatment may be effected prophylactically (prior to infection).

The terms “individual” and “subject” are used interchangeably herein to refer to any member of the subphylum cordata, including, without limitation, humans and other primates (including non-human primates such as chimpanzees and other apes and monkey species); farm animals such as cattle, sheep, pigs, goats and horses; domestic mammals such as dogs and cats; laboratory animals including rodents such as mice, rats and guinea pigs; birds (including domestic, wild and game birds such as chickens, turkeys and other gallinaceous birds, ducks, geese); fish; and the like. The terms do not denote a particular age. Thus, both adult and newborn individuals are intended to be covered. The methods described herein are intended for use in any of the above vertebrate species, since the immune systems of all of these vertebrates operate similarly.

### B. General Methods

Antigens for use with the present invention are preferably derived or obtained from pathogens that enter the tissues of a vertebrate via a mucosal surface. Alternatively the antigens may be synthetic antigens that induce antibodies (especially IgA antibodies) that are directed to, or are specific for a pathogen that enters the body via a mucosal surface. Adjuvants, or adjuvant combinations for use with the present invention preferably contain or are derived from a bacterial exotoxin from the family of bacterial ADP-ribosylating exotoxins (“bAREs”), and more preferably contain or are derived from a cholera toxin (CT). The antigens are administered to a target skin site and are delivered in a particulate form, preferably via a powder injection technique. The adjuvants may likewise be administered to the skin, e.g., in a particulate form and preferably by a powder injection technique.

Antigens

Suitable viral antigens include, but are not limited to, antigens or polynucleotide sequences encoding antigens from the hepatitis family of viruses, including hepatitis A virus (HAV), hepatitis B virus (HBV), hepatitis C virus (HCV), the delta hepatitis virus (HDV), hepatitis E virus (HEV) and hepatitis G virus (HGV). By way of example, the viral genomic sequence of HCV is known, as are methods for obtaining the sequence. See, e.g., International Publication Nos. WO 89/04669; WO 90/11089; and WO 90/14436. The HCV genome encodes several viral proteins, including E1 and E2. See, e.g., Houghton et al. (1991) Hepatology 14:381-388. The sequences encoding each of these proteins, as well as antigenic fragments thereof, will find use in the present methods. Similarly, the coding sequence for the 6-antigen from HDV is known (see, e.g., U.S. Pat. No. 5,378,814). Peptide/protein antigens encoded by these sequences are either known or readily obtainable.

In like manner, a wide variety of proteins from the herpesvirus family can be used as antigens in the present invention, including proteins derived from herpes simplex virus (HSV) types 1 and 2, such as HSV-1 and HSV-2 glycoproteins gB, gD and gH; antigens from varicella zoster virus (VZV), Epstein-Barr virus (EBV) and cytomegalovirus (CMV) including CMV gB and gH; and antigens from other human herpesviruses such as HHV6 and HHV7. (See, e.g. Chee et al. (1990) Cytomegaloviruses (J. K. McDougall, ed., Springer-Verlag, pp. 125-169; McGeoch et al. (1988) J. Gen. Virol. 69:1531-1574; U.S. Pat. No. 5,171,568; Baer et al. (1984) Nature 310:207-211; and Davison et al. (1986) J. Gen. Virol. 67:1759-1816.)

Human immunodeficiency virus (HIV) antigens, such as gp120 molecules for a multitude of HIV-1 and HIV-2 isolates, including members of the various genetic subtypes of HIV, are known and reported (see, e.g., Myers et al., Los Alamos Database, Los Alamos National Laboratory, Los Alamos, N. Mex. (1992); and Modrow et al. (1987) J. Virol. 61:570-578) and antigens derived from any of these isolates will find use in the present methods. Furthermore, the invention is equally applicable to other immunogenic proteins derived from any of the various HIV isolates, including any of the various envelope proteins such as gp160 and gp41, gag antigens such as p24gag and p55gag, as well as proteins derived from the pol, env, tat, vif, rev, nef, vpr, vpu and LTR regions of HIV.

Antigens derived or obtained from other viruses will also find use in the claimed methods, such as without limitation, proteins from members of the families Picornaviridae (e.g., polioviruses, rhinoviruses, etc.); Caliciviridae; Togaviridae (e.g., rubella virus, dengue virus, etc.); Flaviviridae; Coronaviridae; Reoviridae (e.g., rotavirus, etc.); Bimaviridae; Rhabodoviridae (e.g., rabies virus, etc.); Orthomyxoviridae (e.g., influenza virus types A, B and C, etc.); Filoviridae; Paramyxoviridae (e.g., mumps virus, measles virus, respiratory syncytial virus, parainfluenza virus, etc.); Bunyaviridae; Arenaviridae; Retroviradae (e.g., HTLV-I; HTLV-II; HIV-1 (also known as HTLV-III, LAV, ARV, hTLR, etc.)), including but not limited to antigens from the isolates HIV_{IIIb}, HIV_{SF2}, HIV_{LAV}, HIV_{LA1}, HIV_{MN}); HIV-1_{CM235}, HIV-1_{US4}; HIV-2, among others; simian immunodeficiency virus (SIV); Papillomavirus, the tick-bourne encephalitis viruses; and the like. See, e.g. Virology, 3rd Edition (W. K. Joklik ed. 1988); Fundamental Virology, 2nd Edition (B. N. Fields and D. M. Knipe, eds. 1991), for a description of these and other viruses.

Preferably, viral antigens used in the present invention are obtained or derived from a viral pathogen that typically enters the body via a mucosal surface and is known to cause or is associated with human disease, such as, but not limited to, HIV (AIDS), influenza viruses (Flu), herpes simplex viruses (genital infection, cold sores, STDs), rotaviruses (diarrhea), parainfluenza viruses (respiratory infections), poliovirus (poliomyelitis), respiratory syncytial virus (respiratory infections), measles and mumps viruses (measles, mumps), rubella virus (rubella), and rhinoviruses (common cold).

Suitable bacterial and parasitic antigens are obtained or derived from known causative agents responsible for diseases including, but not limited to, Diptheria, Pertussis, Tetanus, Tuberculosis, Bacterial or Futigal Pneumonia, Otitis Media, Gonnorhea, Cholera, Typhoid, Meningitis, Mononucleosis, Plague, Shigellosis or Salmonellosis, Legionaire's Disease, Lyme Disease, Leprosy, Malaria, Hookworm, Onchocerciasis, Schistosomiasis, Trypamasomialsis, Lesmaniasis, Giardia, Amoebiasis, Filariasis, Borelia, and Trichinosis. Still further antigens can be obtained or derived from unconventional viruses such as the causative agents of kuru, Creutzfeldt-Jakob disease (CJD), scrapie, transmissible mink encephalopathy, and chronic wasting diseases, or from proteinaceous infectious particles such as prions that are associated with mad cow disease.

Preferably, bacterial antigens used in the present invention are obtained or derived from a bacterial pathogen that typically enters the body via a mucosal surface and causes or is associated with human disease, such as, but not limited to, Streptococcus (pneumonia, throat infection, otitis media), Haemophilus influenza (otitis media), Moraxella cartarrhalis (otitis media), Neisseria gonnorhoeae (gonnorhea), Vibrio cholerae (cholera, diarrhea), Salmonella (enteric infections, diarrhea), Escherichia coli (enteric infections, genital infections), Shigella (shigellosis), Mycobacterium tuberculosis (tuberculosis), Neisseria meningitidis (meningitis), Mycoplasma pneumoniae (respiratory infections), and Chlamydia (pneumonia, genital infections).

Mucosal immunity may also be important for vaccines or compositions for use in treating or preventing allergic conditions or disorders resulting from inhaled or orally encountered allergens. In this regard, mucosal antibodies (IgA and/or IgG antibodies) can bind and clear such antigens before they can bring about an allergic condition. Classes of suitable allergens for use in the present invention thus include, but are not limited to, pollens, animal dander, grasses, molds, dusts, antibiotics, stinging insect venoms, and a variety of environmental (including chemicals and metals), drug and food allergens. Common tree allergens include pollens from cottonwood, popular, ash, birch, maple, oak, elm, hickory, and pecan trees; common plant allergens include those from rye, ragweed, English plantain, sorrel-dock and pigweed; plant contact allergens include those from poison oak, poison ivy and nettles; common grass allergens include Timothy, Johnson, Bermuda, fescue and bluegrass allergens; common allergens can also be obtained from molds or fungi such as Altemaria, Fusarium, Hormodendrum, Aspergillus, Micropolyspora, Mucor and thermophilic actinomycetes; penicillin and tetracycline are common antibiotic allergens; epidermal allergens can be obtained from house or organic dusts (typically fungal in origin), from insects such as house mites (dermatphagoides pterosinyssis), or from animal sources such as feathers, and cat and dog dander; common food allergens include milk and cheese (diary), egg, wheat, nut (e.g., peanut), seafood (e.g., shellfish), pea, bean and gluten allergens; common environmental allergens include metals (nickel and gold), chemicals (formaldehyde, trinitrophenol and turpentine), Latex, rubber, fiber (cotton or wool), burlap, hair dye, cosmetic, detergent and perfume allergens; common drug allergens include local anesthetic and salicylate allergens; antibiotic allergens include penicillin and sulfonamide allergens; and common insect allergens include bee, wasp and ant venom, and cockroach calyx allergens. Particularly well characterized allergens include, but are not limited to, the major and cryptic epitopes of the Der p I allergen (Hoyne et al. (1994) Immunology 83:190-195), bee venom phospholipase A2 (PLA) (Akdis et al. (1996) J. Clin. Invest. 98:1676-1683), birch pollen allergen Bet v 1 (Bauer et al. (1997) Clin. Exp. Immunol. 107:536-541), and the multi-epitopic recombinant grass allergen rKBG8.3 (Cao et al. (1997) Immunology 90:46-51). These and other suitable allergens are commercially available and/or can be readily prepared as extracts following known techniques.

Antigens for use with the present invention can be obtained or produced using a variety of methods known to those of skill in the art. In particular, the antigens can be isolated directly from native sources, using standard purification techniques. Alternatively, the antigens can be produced recombinantly using known techniques. See, e.g., Sambrook, Fritsch & Maniatis, Molecular Cloning: A Laboratory Manual, Vols. I, II and III, Second Edition (1989); DNA Cloning, Vols. I and II (D. N. Glover ed. 1985). Antigens for use herein may also be synthesized, based on described amino acid sequences, via chemical polymer syntheses such as solid phase peptide synthesis. Such methods are known to those of skill in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis.

If desired, polynucleotide sequences coding for the above-described antigens, can be obtained using recombinant methods, such as by screening cDNA and genomic libraries from cells expressing the gene, or by deriving the gene from a vector known to include the same. Furthermore, the desired gene can be isolated directly from cells and tissues containing the same, using standard techniques, such as phenol extraction and PCR of cDNA or genomic DNA. See, e.g., Sambrook et al., supra, for a description of techniques used to obtain and isolate DNA. Polynucleotide sequences can also be produced synthetically, rather than cloned.

Yet another convenient method for isolating specific nucleic acid molecules is by the polymerase chain reaction (PCR). Mullis et al. (1987) Methods Enzymol. 155:335-350. This technique uses DNA polymerase, usually a thermostable DNA polymerase, to replicate a desired region of DNA. The region of DNA to be replicated is identified by oligonucleotides of specified sequence complementary to opposite ends and opposite strands of the desired DNA to prime the replication reaction. The product of the first round of replication is itself a template for subsequent replication, thus repeated successive cycles of replication result in geometric amplification of the DNA fragment delimited by the primer pair used.

Once obtained, the polynucleotide sequences can be expressed in mammalian, bacterial, yeast, or insect expression systems to provide suitable antigen preparations, or used per se as a nucleic acid antigen composition (e.g., for nucleic acid immunization techniques).

In whatever form the antigen is to be used (peptide, whole organism, nucleic acid), the antigen of interest is formed into a particulate composition as described herein and administered to a subject, generally along with an adjuvant which either serves to enhance the mucosal immune response to the antigen, or directs a mucosal immune response against the antigen. Thus, the particulate composition will be delivered to the skin but, surprisingly, will elicit the generation of a mucosal immune response at one or more mucosal surfaces. It is preferred that the particular transdermal delivery system used perforates the stratum corneum, such as a powder injection system. It is also preferred that the adjuvant is delivered to the skin, typically by a transdermal delivery method that also perforates the stratum corneum. As explained above, the adjuvant can be present in the same or a separate composition as the antigen, and can be delivered simultaneously with the antigen or vaccine composition, or prior or subsequent to antigen delivery. Additionally, the adjuvant can be administered to the same or to a different site.

Adjuvants

The present invention may effectively be used with any suitable adjuvant or combination of adjuvants. For example, suitable adjuvants include, without limitation, adjuvants formed from aluminum salts (alum), such as aluminum hydroxide, aluminum phosphate, aluminum sulfate, etc; oil-in-water and water-in-oil emulsion formulations, such as Complete Freunds Adjuvants (CFA) and Incomplete Freunds Adjuvant (IFA); adjuvants formed from bacterial cell wall components such as adjuvants including lipopolysaccharides (e.g., lipid A or monophosphoryl lipid A (MPL), Imoto et al. (1985) Tet. Lett. 26:1545-1548), trehalose dimycolate (TDM), and cell wall skeleton (CWS); heat shock protein or derivatives thereof, adjuvants derived from ADP-ribosylating bacterial toxins, including diphtheria toxin (DT), pertussis toxin (PT), cholera toxin (CT), the E. coli heat-labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile, Staphylococcus aureus EDIN, and ADP-ribosylating bacterial toxin mutants such as CRM₁₉₇, a non-toxic diphtheria toxin mutant (see, e.g., Bixler et al. (1989) Adv. Exp. Med. Biol. 251:175; and Constantino et al. (1992) Vaccine); saponin adjuvants such as Quil A (U.S. Pat. No. 5,057,540), or particles generated from saponins such as ISCOMs (immunostimulating complexes); chemokines and cytokines, such as interleukins (e.g., IL-1, IL-2, IL-4, IL-5, IL-6, IL-7, IL-8, IL-12, etc.), interferons (e.g., gama interferon), macrophage colony stimulating factor (M-CSF), tumor necrosis factor (TNF), defensins 1 or 2, RANTES, MIP1-α and MIP-2, etc; muramyl peptides such as N-acetyl-muramyl-L-threonyl-D-isoglutamine (thr-MDP), N-acetyl-normuramyl-^{L}-alanyl-^{D}-isoglutamine (nor-MDP), N-acetylmuramyl-^{L}-alanyl-^{D}-isoglutaminyl-^{L}-alanine-2-(1′-2′-dipalmitoyl-sn-glycero-3 huydroxyphosphoryloxy)-ethylamine (MTP-PE) etc.; adjuvants derived from the CpG family of molecules, CpG dinucleotides and synthetic oligonucleotides which comprise CpG motifs (see, e.g., Krieg et al. Nature (1995) 374:546, Medzhitov et al. (1997) Curr. Opin. Immunol. 9:4-9, and Davis et al. J. Immunol. (1998) 160:870-876) such as TCCATGACGTTCCTGATGCT (SEQ ID NO: 1) and ATCGACTCTCGAGCGTTCTC (SEQ ID NO: 2); and synthetic adjuvants such as PCPP (Poly[di(carboxylatophenoxy)phos-phazene) (Payne et al. Vaccines (1998) 16:92-98). Such adjuvants are commercially available from a number of distributors such as Accurate Chemicals; Ribi Immunechemicals, Hamilton, Mont.; GIBCO; Sigma, St. Louis, Mo. Preferred adjuvants are those derived from ADP-ribosylating bacterial toxins, with cholera toxin and heat labile toxins being most preferred. Oligonucleotides containing a CpG motif are also preferred.

The adjuvant may delivered individually or delivered in a combination of two or more adjuvants. In this regard, combined adjuvants may have an additive or a synergistic effect in promoting a mucosal immune response. A synergistic effect is one where the result achieved by combining two or more adjuvants is greater than one would expect than by merely adding the result achieved with each adjuvant when administered individually. A preferred adjuvant combination is an adjuvant derived from an ADP-ribosylating bacterial toxin and a synthetic oligonucleotide comprising a CpG motif. A particularly preferred combination comprises cholera toxin and the oligonucleotide ATCGACTCTCGAGCGTTCTC (SEQ ID NO: 2).

Unfortunately, a majority of the above-referenced adjuvants are known to be highly toxic, and are thus generally considered too toxic for human use. It is for this reason that the only adjuvant currently approved for human usage is alum, an aluminum salt composition. Nevertheless, a number of the above adjuvants are commonly used in animals and thus suitable for numerous intended subjects, and several are undergoing preclinical and clinical studies for human use. However, as discussed herein above, the adjuvants are preferably rendered into particulate form for transdermal delivery using a powder injection method. Surprisingly, it has been found that adjuvants which are generally considered too toxic for human use may be rendered into particulate form and administered with a powder injection technique without concomitant toxicity problems. Without being bound by a particular theory, it appears that delivery of adjuvants to the skin, using transdermal delivery methods (powder injection), allows interaction with Langerhans cells in the epidermal layer and dendritic cells in the cutaneous layer of the skin. These cells are important in initiation and maintenance of an immune response. Thus, an enhanced adjuvant effect can be obtained by targeting delivery to or near such cells. Moreover, transdermal delivery of adjuvants in the practice of the invention may avoid toxicity problems because (1) the top layers of the skin are poorly vascularized, thus the amount of adjuvant entering the systemic circulation is reduced which reduces the toxic effect; (2) skin cells are constantly being sloughed, therefore residual adjuvant is eliminated rather than absorbed; and (3) substantially less adjuvant can be administered to produce a suitable adjuvant effect (as compared with adjuvant that is delivered using conventional techniques such as intramuscular injection).

Once selected, one or more adjuvant can be provided in a suitable pharmaceutical form for parenteral delivery, the preparation of which forms are well within the general skill of the art. See, e.g., Remington's Pharmaceutical Sciences (1990) Mack Publishing Company, Easton, Pa., 18th edition. Alternatively, the adjuvant can be rendered into particulate form as described in detail below. The adjuvant(s) will be present in the pharmaceutical form in an amount sufficient to bring about the desired effect, that is, either to enhance the mucosal response against the coadministered antigen of interest, and/or to direct a mucosal immune response against the antigen of interest. Generally about 0.1 μg to 1000 μg of adjuvant, more preferably about 1 μg to 500 μg of adjuvant, and more preferably about 5 μg to 300 μg of adjuvant will be effective to enhance an immune response of a given antigen. Thus, for example, for CpG, doses in the range of about 0.5 to 50 μg, preferably about 1 to 25 μg, and more preferably about 5 to 20 μg, will find use with the present methods. For cholera toxin, a dose in the range of about 0.1 μg to 50 μg, preferably about 1 μg to 25 μg, and more preferably about 5 μg to 15 μg will find use herein. Similarly, for alum or PCPP, a dose in the range of about 2.5 μg to 500 μg, preferably about 25 to 250 μg, and more preferably about 50 to 150 μg, will find use herein. For MPL, a dose in the range of about 1 to 250 μg, preferably about 20 to 150 μg, and more preferably about 40 to 75 μg, will find use with the present methods.

Doses for other adjuvants can readily be determined by one of skill in the art using routine methods. The amount to administer will depend on a number of factors including the coadministered antigen, as well as the ability of the adjuvant to act as stimulator of a mucosal immune response.

Particulate Compositions

Once obtained, the antigen of interest (as well as the selected adjuvant) can be formulated as a particulate composition. More particularly, formulation of particles comprising the antigen and/or adjuvant of interest can be carried out using standard pharmaceutical formulation chemistries and methodologies all of which are readily available to the reasonably skilled artisan. For example, one or more antigen and/or adjuvant can be combined with one or more pharmaceutically acceptable excipient or vehicle to provide an antigen, adjuvant, or vaccine composition. Auxiliary substances, such as wetting or emulsifying agents, pH buffering substances, and the like, may be present in the excipient or vehicle. These excipients, vehicles and auxiliary substances are generally pharmaceutical agents that do not themselves induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that an antigen composition will contain a pharmaceutically acceptable carrier that serves as a stabilizer, particularly for peptide, protein or other like antigens. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, carriers, stabilizers and other auxiliary substances is available in REMINGTON'S PHARMACEUTICAL SCIENCES (Mack Pub. Co., N.J. 1991), incorporated herein by reference.

The formulated compositions will include an amount of the antigen of interest which is sufficient to mount an immunological response, as defined above. An appropriate effective amount can be readily determined by one of skill in the art. Such an amount will fall in a relatively broad range, generally within the range of about 0.1 μg to 25 mg or more of the antigen of interest, and specific suitable amounts can be determined through routine trials. The compositions may contain from about 0.1% to about 99.9% of the antigen. If an adjuvant is included in the composition, or the methods are used to provide a particulate adjuvant composition, the adjuvant will be present in a suitable amount as described above. The compositions are then prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in commonly owned International Publication No. WO 97/48485, incorporated herein by reference.

Single unit dosages or multidose containers, in which the particles may be packaged prior to use, can comprise a hermetically sealed container enclosing a suitable amount of the particles comprising the antigen of interest and/or the selected adjuvant (e.g., the vaccine composition). The particulate compositions can be packaged as a sterile formulation, and the hermetically sealed container can thus be designed to preserve sterility of the formulation until use in the methods of the invention. If desired, the containers can be adapted for direct use in the a needleless syringe system.

The container in which the particles are packaged can further be labelled to identify the composition and provide relevant dosage information. In addition, the container can be labelled with a notice in the form prescribed by a governmental agency, for example the Food and Drug Administration, wherein the notice indicates approval by the agency under Federal law of the manufacture use or sale of the antigen, adjuvant (or vaccine composition) contained therein for human administration.

The particulate compositions (comprising the antigen of interest and/or a selected adjuvant) can then be administered using a transdermal delivery technique. Preferably, the particulate compositions will be delivered via a powder injection method, e.g., delivered from a needleless syringe system such as those described in commonly owned International Publication Nos. WO 94/24263, WO 96/04947, WO 96/12513, and WO 96/20022 all of which are incorporated herein by reference. Delivery of particles from such needleless syringe systems is practiced with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec, or greater. With appropriate gas pressure, particles having an average diameter of 10-70 μm can be accelerated through the nozzle at velocities approaching the supersonic speeds of a driving gas flow.

If desired, these needleless syringe systems can be provided in a preloaded condition containing a suitable dosage of the particles comprising the antigen of interest and/or the selected adjuvant. The loaded syringe can be packaged in a hermetically sealed container, which may further be labeled as described above.

Coated Particles

Alternatively, if intracellular delivery is desired, the antigens and/or adjuvants can be coated onto suitable carrier particles, e.g., gold or tungsten. If the antigen is to be provided in nucleic acid form, intracellular delivery is preferred. Accordingly, in one embodiment of the invention, the antigens, polynucleotides encoding antigens (e.g., DNA vaccines) and/or adjuvants are delivered using carrier particles. Particle-mediated methods for delivering such vaccine preparations are known in the art. More particularly, once prepared and suitably purified, antigens, nucleic acid molecules encoding antigens and/or adjuvants can be coated onto carrier particles (e.g., ballistic core carriers) using a variety of techniques known in the art. Carrier particles are selected from materials which have a suitable density in the range of particle sizes typically used for intracellular delivery from a gene gun device. The optimum carrier particle size will, of course, depend on the diameter of the target cells.

Any suitable carrier particle can be used, for example, particles formed from polymers or metals (e.g., tungsten, gold, platinum and iridium); however, tungsten and gold carrier particles are preferred. Tungsten particles are readily available in average sized of 0.5 to 2.0 μm in diameter. Although such particles have optimal density for use in particle-mediated delivery methods, and allow highly efficient coating with DNA, tungsten may potentially be toxic to certain cell types. Gold particles or microcrystalline gold (e.g., gold powder A1570, available from Engelhard Corp., East Newark, N.J.) will also find use with the present methods. Gold particles provide uniformity in size (available from Alpha Chemicals in particle sizes of 1-3 μm, or available from Degussa, South Plainfield, N.J. in a range of particle sizes including 0.95 μm) and reduced toxicity. Microcrystalline gold provides a diverse particle size distribution, typically in the range of 0.5-5 μm. However, the irregular surface area of microcrystalline gold provides for highly efficient coating with nucleic acids, antigens and adjuvants.

A number of methods are known and have been described for coating or precipitating DNA or RNA onto gold or tungsten particles. Such methods generally combine a predetermined amount of gold or tungsten with plasmid DNA, CaCl.sub.2 and spermidine. The resulting solution is vortexed continually during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in particular gene gun instruments.

Peptide antigens and/or adjuvants can be attached to carrier particle by simply mixing the two components in an empirically determined ratio, by ammonium sulfate precipitation or other solvent precipitation methods familiar to those skilled in the art, or by chemical coupling of the peptide to the carrier particle. The coupling of L-cysteine residues to gold has been previously described (Brown et al. (1980) Chemical Society Reviews 9:271-311). Other methods include, for example, dissolving the peptide antigen in absolute ethanol, water, or an alcohol/water mixture, adding the solution to a quantity of carrier particles, and then drying the mixture under a stream of air or nitrogen gas while vortexing. Alternatively, the peptide antigens can be dried onto carrier particles by centrifugation under vacuum. Once dried, the coated particles can be resuspended in a suitable solvent (e.g., ethyl acetate or acetone), and triturated (e.g., by sonication) to provide a substantially uniform suspension.

Following their formation, carrier particles coated with antigen and/or adjuvant preparations are delivered to the target skin site using a particle-mediated delivery technique. Various particle acceleration devices suitable for particle-mediated delivery are known in the art, and are all suited for use in the practice of the invention. Current device designs employ an explosive, electric or gaseous discharge to propel the coated carrier particles toward target cells. The coated carrier particles can themselves be releaseably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of a helium discharge-type particle acceleration apparatus is the PowderJect® XR instrument (PowderJect. Vaccines, Inc., Madison), WI, which instrument is described in U.S. Pat. No. 5,120,657. An electric discharge apparatus suitable for use herein is described in U.S. Pat. No. 5,149,655. The disclosure of all of these patents is incorporated herein by reference.

Single dosages of the coated carrier particles can be provided in a suitable container, for example, provided in a length of tubing which contains a dose of the particles coated on an inner surface thereof. Methods for preparing such containers are described in commonly owned U.S. Pat. Nos. 5,733,600 and 5,780,100, the disclosures of which are incorporated herein by reference.

The particle compositions or coated particles are administered to the individual in a manner compatible with the dosage formulation, and in an amount that will be effective for the purposes of the invention. The amount of the composition to be delivered (e.g., about 0.1 μg to 1 mg, more preferably 1 to 50 μg of the antigen or allergen, depends upon the individual to be tested and the particular antigen(s) or allergen(s) being administered. The exact amount necessary will vary depending on the age and general condition of the individual to be treated, and an appropriate effective amount can be readily determined by one of skill in the art upon reading the instant specification.

Administration and Dosage Schedules

Antigen, adjuvant, or composition(s) containing antigen and/or adjuvant are administered to the subject in a manner compatible with the dosage formulation, and in amounts effective to bring about a desired mucosal immune response. The amount of the antigen to be delivered per administration is, in the case of nucleic acid molecules encoding an antigen, generally in the range of from about 0.001 g to 10 mg, and preferably about 0.01 to 5000 μg of nucleic acid molecule per dose (generally in the range of from 0.5 μg/kg to 100 μg/kg of nucleic acid molecule per dose), and in the case of an antigen molecule (e.g., a peptide, carbohydrate or whole organism) is about 1 μg to 20 mg, and preferably about 1 μg to 5 mg, and more preferably about 10 μg to 3 mg. The exact amount will, of course, depend upon both the subject and the condition to be treated or prevented. More particularly, the exact amount necessary will vary depending on the age and general condition of the individual, and the particular antigen(s) and/or adjuvant(s) selected, as well as other factors. An appropriate effective amount can be readily determined by one of skill in the art upon reading the instant specification and/or can be determined through routine trials.

For powder injection, transdermal delivery is carried out with particles having an approximate size that generally ranges between 0.1 and 250 μm. However, the optimal particle size is usually at least about 10 to 15 μm (the size of a typical cell). Particles larger than about 250 μm can also be delivered from the device, with the upper limitation being the point at which the size of the particles would cause untoward damage to the skin. Other particulate biopharmaceuticals, such as peptide and protein preparations, will generally have an approximate size of 0.1 to about 250 μm, preferably about 0.1 to about 150 μm, and most preferably about 20 to about 60 μm.

The actual distance which the delivered particles will penetrate depends upon particle size (e.g., the nominal particle diameter assuming a roughly spherical particle geometry), particle density, the initial velocity at which the particle impacts the skin surface, and the density and kinematic viscosity of the skin. In this regard, optimal particle densities for use in needleless injection generally range between about 0.1 and 25 g/cm³, and optimal bulk densities range from about 0.5 and 3.0 g/cm³. Injection velocities generally range between about 100 and 3,000 m/sec.

If desired, the adjuvant or compositions containing adjuvant can be prepared as injectables, typically either as liquid solutions or suspensions, or as solid forms suitable for solution or suspension in liquid vehicles prior to injection. The adjuvant may also be emulsified or even encapsulated in, e.g., liposome vehicles. Adjuvant compositions suitable for topical administration can also be prepared, for example in the form of pharmaceutically acceptable topical vehicles such as ointments, emulsions, gels, and creams or lotions. Other suitable pharmaceutical forms and modes of administration will readily occur to the ordinarily skilled artisan upon reading this specification.

Dosage treatment may be a single dose schedule or a multiple dose schedule. For vaccine compositions, a multiple dose schedule is one in which a primary course of vaccination may be with 1-10 separate doses, followed by other doses given at subsequent time intervals, chosen to maintain and/or reinforce the immune response, for example at 1-4 months for a second dose, and if needed, a subsequent dose (s) after several months. The dosage regimen will also, at least in part, be determined by the need of the subject and be dependant on the judgment of the practitioner. Furthermore, if prevention of disease is desired, the compositions are generally administered prior to primary infection with the pathogen of interest. If treatment is desired, e.g., the reduction of symptoms or recurrences, the compositions are generally administered subsequent to primary infection.

### C. Examples

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

General Materials and Methods

Vaccine formulation: Vaccines can be formulated in powder form using a number of particle formation processes. Excipients, such as trehalose, sucrose, agarose, mannitol, or a mixture of sugar excipients are preferably used in the formulations to provide for stabilization (of the antigen(s) and/or adjuvant(s)) and enhanced particle characteristics (e.g., enhanced hardness, density). Standard particle preparation processes include air-drying, freeze-drying, spray-coating, spray-drying, spray-freeze drying, arid supercritical fluid technology.

The following is an example of air-drying a vaccine composition. First, the antigen (such as diphtheria toxoid, influenza vaccine, etc.) was combined with adjuvant (e.g. cholera toxin, synthetic oligonucleotide containing CpG motif, etc.), then mixed with a trehalose/water solution. The solution was gently mixed, poured into glass petri dishes and allowed to air-dry for two days under a fume hood, after which it was further dried in a desiccator (Nalgene™ Plastic desiccator) then purged with N.sub.2 gas for another day. The dried solid was collected by scraping and was ground with mortar and pestle. Finally, dry powder was weighed and the amount of dry material for each dose was determined by dividing the total weight by the number of doses formulated. Particle size distribution of the reformulated vaccine composition was measured, and a broad size range of particles was used (e.g., 1-100 mm). An appropriate amount of the powdered vaccine composition was measured using a balance and loaded into a particle cassette. Typically, each dose required about 1-2 mg of dry mass. The variation in weighing was 10% or less.

Device: The powder injection technology employs a helium-powered PowderJect® ND powder injection device (needleless syringe) as previously described (see, e.g., U.S. Pat. No. 5,899,880 to Bellhouse, et al.). The powder injection device that was used to conduct the present studies is a reusable research model obtained from PowderJect Vaccines, Inc. (Madison, Wis.). The instrument is approximately 15 cm in length and comprises an actuation button, a helium gas chamber, a vaccine cassette (containing the particulate vaccine composition), a nozzle, and a silencer. In use, the stainless steel gas chamber is filled with approximately 5 ml of medical grade helium gas to 50 bar pressure. Upon actuation, the released helium gas ruptures the membranes of the trilaminate cassette and accelerates the vaccine particles to sufficiently high velocity such that the particles perforate the stratum corneum and are delivered into the epidermis. The helium gas is reflected off the skin and exhausted through the vented silencer. The depth of particle penetration depends upon the powder formulation, the particle size, the gas pressure, and the mechanics of the device. All of these parameters can be experimentally optimized to deliver powders through the stratum corneum of different thickness.

Mice and vaccination: Female Balb/C mice or Swiss Webster mice. 7 weeks of age were purchased from HSD and acclimatised for 1 week at a mouse facility before vaccination. Mice were anesthetized by an intraperitoneal injection of 100 mg/kg ketamine mixed with 10 mg/kg xylazine, and the abdominal skin was depiliated by shaving. The powder injection device was gently pressed against the vaccination site and actuated. A typical immunization regime consisted of two vaccinations four weeks apart, with blood collection via retro-orbital bleeding under anesthesia prior to each vaccination and two weeks post boost. For transcutaneous immunization (TCI) procedures, the abdominal skin of the anesthetized mice was shaved, and the liquid vaccine compositions were applied to the shaved area and left for 1 hour before being washed off with warm tap water.

ELISA: Antibody response to the various vaccine compositions was determined by a standard ELISA procedure. More particularly, a 96-well plate was coated with 0.1 μg (diphtheria toxoid) or 1 μg (influenza) of detecting antigen in PBS (per well) and maintained overnight at 4.degree. C. Plates were washed 3 times with TBS containing 0.1% Brij-35, and incubated with test sera diluted in PBS containing 1% BSA (diphtheria toxoid) or 5% dry milk (influenza) for 1.5 hr. A serum standard containing a high level of antibodies to specific antigen, was added to each plate and used to standardize the titer in the final data analysis. The plates were then washed and incubated with biotin-labelled goat antibodies specific for mouse immunoglobulin IgG or specific IgG subclasses (1:8,000 in PBS, Southern Biotechnology) for 1 hr at room temperature. Following three additional washes, the ELISA plates were incubated with strepavidin-horseradish peroxidase conjugates (1:8,000 in PBS, Southern Biotechnology) for 1 hr at room temperature. Finally, plates were washed and developed with TMB substrate (kit from Bio-Rad, Richmond, Calif.). Endpoint titers of the test sera were determined using the Softmax Pro 4.1 program (Molecular Devices) as the calibrated highest dilution with an A₄₅₀ that exceeded the mean background by 0.1. Mean background absorbance was determined by wells that received all reagents except for test sera.

### Example 1

### Induction of Mucosal Immune Response to Diphtheria Toxoid by Skin Immunization

In order to determine whether powder injection delivery of a vaccine composition to skin would induce a mucosal antibody response, the following study was carried out. 5 μg of diphtheria toxoid and 10 μg of cholera toxin were combined with a trehalose excipient and formulated into a powdered vaccine composition as described above. The powdered vaccine composition was then delivered via powder injection to immunize Balb/C mice. Immunizations were given on days 0 and 28 of the study. Serum and saliva was collected on day 42. Diphtheria toxoid-specific antibody titers were then determined in an ELISA procedure.

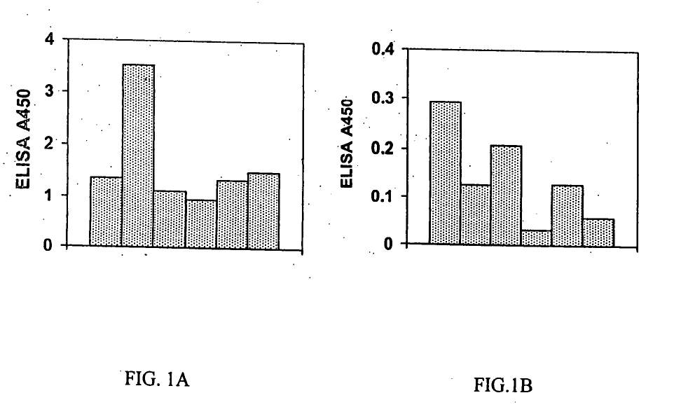

In addition to the induction of an antigen-specific serum IgG titer (data not shown), powder injection of the diphtheria toxoid to skin induced both IgG (FIG. 1A) and IgA (FIG. 1B) antibodies to diphtheria toxoid in saliva. It is known that conventional needle and syringe injection to deeper tissues (e.g. muscle) usually does not induce IgA antibodies in the saliva, as contrasted with the present epidermal powder injection immunization which can elicit a mucosal (IgA antibody) response.

### Example 2

### A Comparison of Mucosal Antibody Responses Obtained by Either Powder Injection or TCI Administration Procedures

In order to compare the ability of vaccine compositions to elicit a mucosal antibody response when delivered to the skin by either powder injection or TCI administration, the following study was carried out. Vaccine compositions were formulated in either particulate (for powder injection) or liquid form (for TCI administration) and contained 5 μg of diphtheria toxoid combined with 5 μg of cholera toxin. The compositions were used to immunize Balb/C mice by either method, where immunizations were given on weeks 0 and 4 of the study. Serum and mucosal tissue samples were collected on week 6. The mucosal tissue samples (trachea, lung, vagina, small intestine, Peyer's patch, and mesenteric lymph node) were cultured in vitro for 7 days. Tissue culture supernatants and serum were assayed by ELISA for antigen-specific responses.

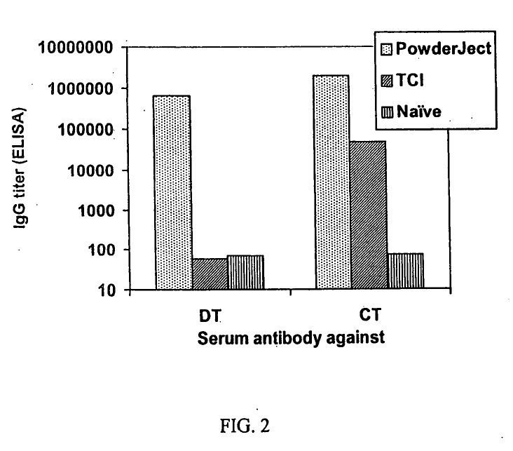

The results of the study are depicted in FIGS. 2-4. First, as can be seen in FIG. 2, powder injection elicited high levels of serum IgG antibodies to both diphtheria toxoid and cholera toxin, whereas TCI administration of the same vaccine composition only elicited moderate levels of serum IgG to cholera toxin, and no detectable IgG antibodies to the co-administered diphtheria toxoid (FIG. 2).

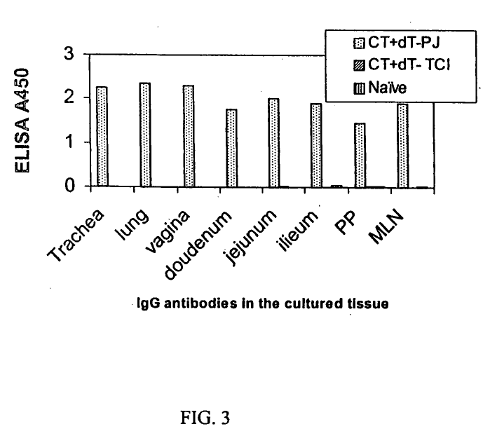

In addition, as shown in FIG. 3, a high level of IgG antibodies were consistently found in the mucosal tissue culture supernatant from the mice that were immunized via powder injection. These antibodies were likely produced by antibody-secreting cells residing in the mucosal tissue samples during their 7-day in vitro culture (all serum antibodies were removed prior to initiating the culture by way of extensive washing with buffer and culture medium). By contrast, TCI immunization of mice with the same vaccine composition did not lead to any detectable antibodies in any of the cultured mucosal tissues (FIG. 3). These data suggest that powder injection of a vaccine composition to skin successfully results in the generation of antigen-specific antibody secreting cells that home to disseminated mucosal tissues, and important step in generating mucosal immunity against the antigen of interest. These data also-suggest that powder injection can be used to provide successful immunization status in a subject using doses of antigen and adjuvant that would not work by TCI.

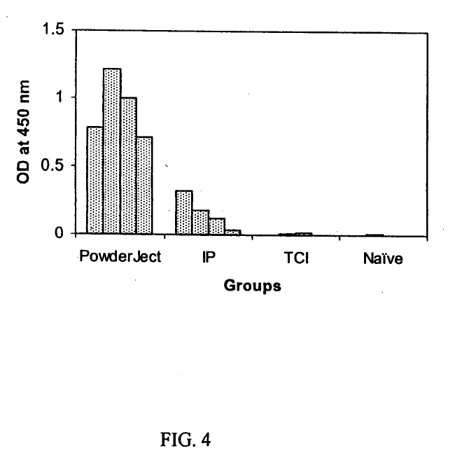

Finally, the successful induction of mucosal immunity by powder injection to the skin is also shown by examining the antibodies in the mucosal secretions. Referring now to FIG. 4, powder injection, but not TCI administration, was found to induce a high level of IgG response in the nasal secretions of immunized mice (FIG. 4). It is well accepted now that parenteral injection (such as the intraperitoneal (IP) injection control in this study) does not generate an antibody response in the nasal mucosa. The presence of elevated IgG in the nasal secretion (as seen with only the powder injection) is an indication of successful mucosal immune response. Furthermore, high levels of IgG antibodies were also found in the vaginal secretion, lung lavage and fecal samples of mice immunized via powder injection to the skin (data not shown).

### Example 3

### Induction of Mucosal Immune Response to Influenza Virus by Skin Immunization

In order to establish that powder injection of vaccine compositions to skin can elicit a mucosal immune response to other antigens, the following study was carried out. An inactivated influenza vaccine was obtained from a commercial source. Since inactivated influenza viruses are 80-120 nm particles, TCI is unlikely to deliver such a vaccine through intact skin because of the relative impermeability of the stratum corneum.

More particularly, 5 μg of inactivated influenza virus (strain Aichi/68, H.sub.3N.sub.2) was formulated as either a powder or as a liquid vaccine, each composition further containing 5 μg of cholera toxin adjuvant. The vaccine compositions were administered to Balb/C mice by either powder injection or TCI administration. Immunization was given on weeks 0 and 4 of the study. Mucosal secretions were collected on week 6 for antibody analysis. Mucosal tissue fragment samples were collected on week 6 for in vitro culture and antibody analysis.

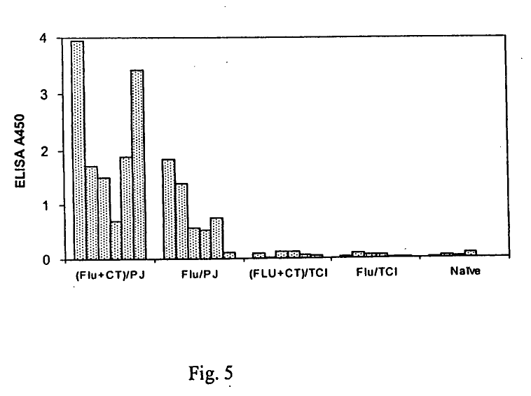

The results of the study are depicted in FIGS. 5-7. Initially, it was found that powder injection delivery of the vaccine composition (cholera toxin (CT) formulated with influenza virus) elicited influenza-specific IgA responses in the trachea based on the in vitro tissue culture assay (see FIG. 5). Furthermore, the cholera toxin adjuvant enhances the mucosal IgA response in the mice immunized by powder injection (see FIG. 5 for a comparison between powder injection of flu alone and powder injection of flu formulated with CT). TCI administration of influenza alone or in combination with the cholera toxin adjuvant failed to induce a detectable IgA response against the influenza vaccine in this assay.

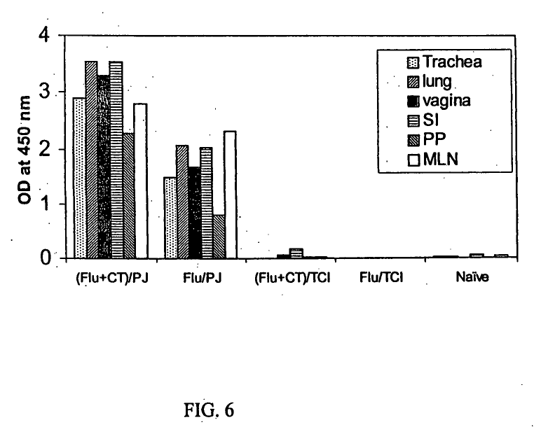

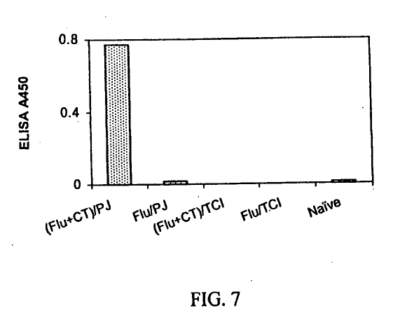

Referring now to FIG. 7, when the nasal secretion was examined, elevated IgG antibodies to the influenza virus were detected by ELISA in the mice receiving vaccine via powder injection. Here again, the cholera toxin adjuvant appears to have enhanced the antibody response (see FIG. 6 for a comparison between powder injection of flu alone and powder injection of flu formulated with CT). TCI administration of either flu alone, or flu combined with the CT adjuvant failed to elicit a detectable antibody response (FIG. 7).

Finally, referring to FIG. 6, it was also found that powder injection of the particulate vaccine compositions elicited flu-specific IgG antibody responses in a variety of different mucosal tissues based on the tissue fragment culture assay. This mucosal immune response was enhanced by formulating the influenza vaccine with the cholera toxin adjuvant (see FIG. 7 for a comparison between powder injection of flu alone and powder injection of flu formulated with the CT adjuvant).

### Example 4

### Induction of Mucosal Immune Response to Influenza Virus by Skin Immunization

The following study was performed in order to determine whether adjuvants other than the CT adjuvant can be used to induce a mucosal immune response when delivered to the skin via a powder injection technique. A secondary purpose of this study was to find out if a combination of the CpG and CT adjuvants would act in synergistic or additive fashion to induce a mucosal response when administered to the skin via powder injection.

The PR8 strain of inactivated influenza virus was used as the antigen of interest in this study. 5 μg of inactivated influenza virus (stain PR8/38, H1N1) was combined with 5 μg of the CT adjuvant (either with or without 10 μg of CpG DNA) or with 10 μg of CpG DNA (CpG sequence: ATCGACTCTCGAGCGTTCTC (SEQ ID NO: 2)) and formulated as particulate (powder) compositions. The vaccine compositions were administered to Balb/C mice using a powder injection device. Immunizations were carried out on weeks 0 and 4 of the study. Mucosal secretions and sera samples were collected on week 6, and then used for antibody analysis. The mucosal tissue fragments were collected on week 6 for in vitro culture and antibody analysis.

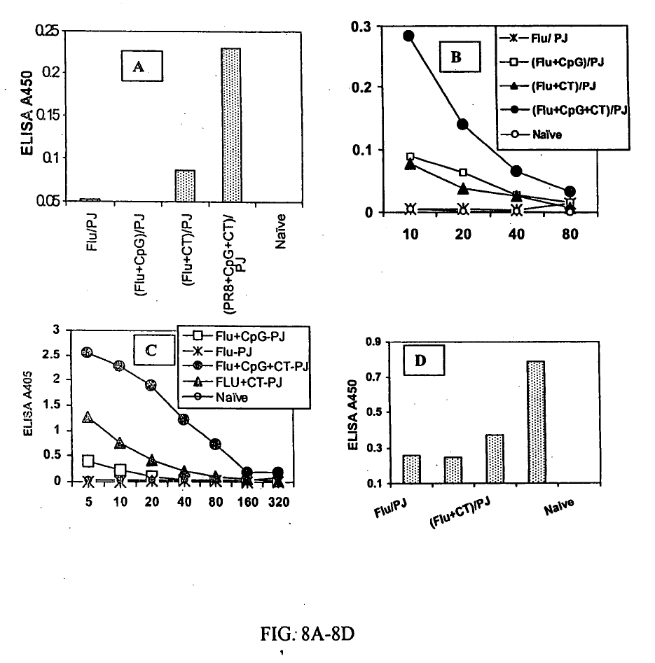

The results of the study are depicted in FIGS. 8-10. Referring now to FIGS. 8A-8D, the influenza vaccine composition formulated with the CT adjuvant provided for a secretory IgA antibody response in nasal wash (FIG. 8A), saliva (FIG. 8B), vaginal wash (FIG. 8C), and fecal extract (FIG. 8D) following powder injection to the skin. This is consistent with the results obtained in Example 3 above. Furthermore, the vaccine composition containing the CpG adjuvant also elicited a mucosal response to the influenza vaccine (FIGS. 8A-8D). However, strikingly, the data demonstrate that the combination of the CpG and CT adjuvants provided for a synergistic effect, that is, the combination was more potent than either adjuvant alone in inducing secretory IgA responses (FIGS. 8A-8D). The combination of the CT and CpG adjuvants provided consistently higher levels of IgA titers in all the mucosal secretions measured (nasal, saliva, vaginal and fecal).

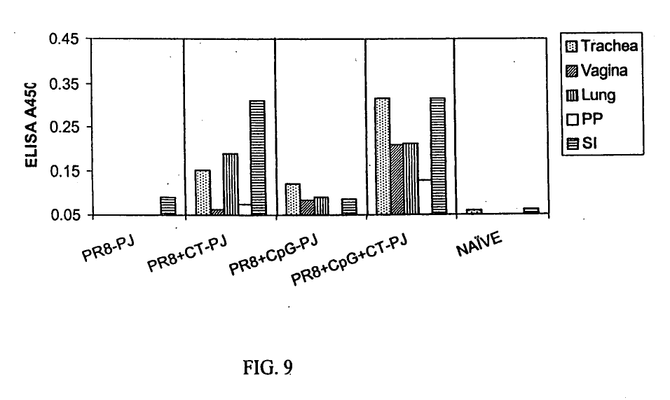

As can be seen in FIG. 9, IgA antibodies were also detected in the supernatants from the in vitro cultured mucosal tissue samples (trachea, vagina, lung, Peyer's patch, and small intestines) obtained from mice receiving vaccine compositions containing either CT or CpG adjuvant. However, consistent with the results reported above, the combination of the CT and CpG adjuvants elicited higher IgA production than either adjuvant alone, as well as higher IgA production than one would expect by merely adding the intensities of the adjuvant effect when those adjuvants were administered individually. These data show that powder injection of the particulate influenza vaccine compositions to skin elicited a disseminated mucosal immune response, and further that the CpG/CT adjuvant combination acts in a synergistic fashion in promoting a mucosal immune response.

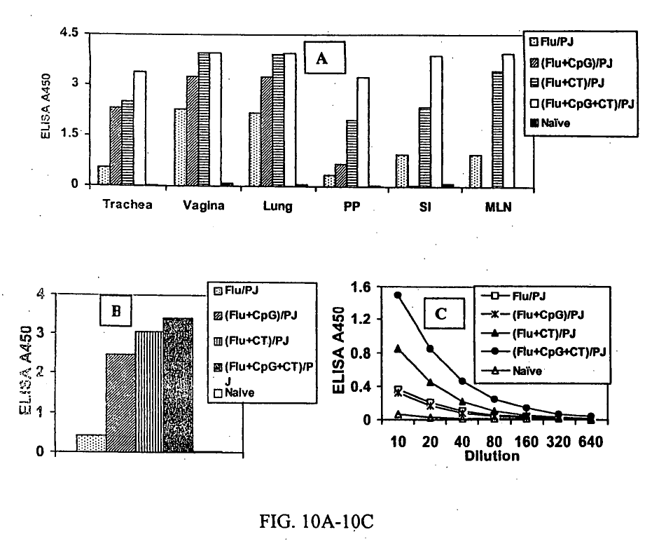

Finally, the IgG titers from fecal extract, saliva, and mucosal tissue cultures were also determined (see the results reported in FIGS. 10A-10C). Elevated IgG titers were noted in these mucosal samples when CT or CpG adjuvant was used, and the combination of adjuvants was again more potent then either one alone in promoting the mucosal IgG response against the flu vaccine.

### Example 5

### Induction of Mucosal Immune Response to Influenza Antigen by Skin Immunization

The following study was performed to assess a number of issues. Initially, the study was carried out to establish that the ability to elicit anti-influenza mucosal immunity via skin administration (as shown in the Examples above) was not strain-specific. In addition, the effect of combining the CpG and CT adjuvants on the ability to induce a mucosal response when administered to the skin via powder injection was revisited.

An influenza vaccine product was obtained from a commercial source (the Strain A/Sydney/5197 (H.sub.3N.sub.2) influenza subunit vaccine product obtained from Swiss Serum Institute, Switzerland). A 1/10th dose of the human influenza vaccine product (corresponding to 1.5 μg of HA antigen) was combined with a trehalose excipient and formulated as a particulate either pet se, in combination with 10 μg of the CT adjuvant, or in combination with 5 μg of the CT adjuvant and 10 μg of CpG DNA (CpG sequence: ATCGACTCTCGAGCGTTCTC (SEQ ID NO: 2)). The particulate vaccine compositions were administered to Balb/C mice (8 mice/group) using a powder injection device. Immunization was carried out on week 0, and blood serum samples were collect on week 4 of the study just prior to a week 4 challenge with a mouse-adapted Aichi/68 (H.sub.3N.sub.2) influenza virus at 10× the LD₅₀ by intranasal delivery. Control mice did not receive any vaccine composition. Influenza-specific IgG titers were then determined in the above-described ELISA procedure.

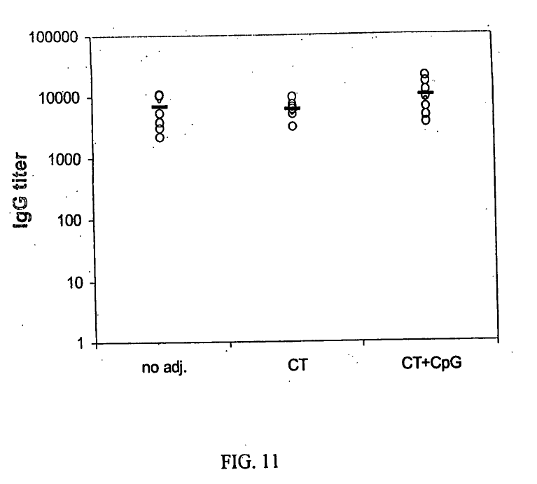

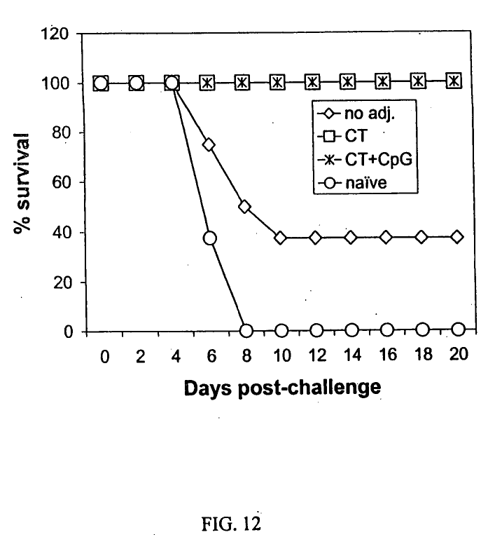

The results of the study are depicted in FIGS. 11-12. With respect to FIG. 11, antibody (IgG) responses were measured by ELISA using the four week post-vaccination serum samples. As can be seen, immunization with the composition containing both CT and CpG adjuvant provided a higher IgG titer. Referring now to FIG. 12, the ability of the various vaccine composition to protect the immunized mice against challenge with a heterologous influenza virus are graphically illustrated. As can be seen, the vaccine compositions containing the 1/10th dose of A/Sydney Strain human vaccine with either the CT adjuvant or the combined CT and CpG adjuvant provided complete protection (100% survival) over the 21 day monitoring period of the challenge study. These results are contrasted with the 100% mortality seen in the naive control group, and the 60% mortality rate seen in the mice receiving the 1/10th dose of A/Sydney Strain human vaccine without adjuvant. The vaccine compositions of the present invention provided protection against mucosal exposure to the challenge virus after having been administered to the skin via needleless powder injection.

### Example 6

### Comparison of Cholera Toxin Toxicity When Administered by Different Routes

Cholera toxin (CT) is one of the most potent immunogens yet identified, inducing strong mucosal responses when used in the methods of the invention. The following study was carried out to assess the relative toxicities of a range of doses of the CT adjuvant when administered directly to mucosal tissue (intranasal application of liquid CT) or administered in particle form to skin via needleless powder injection (via a PowderJect® ND powder injection device). More particularly, the following doses of CT were administered to groups of 8 Balb/c mice by either powder injection or intranasal application: 1 μg, 5 μg, 10 μg, 20 μg, and 50 μg. The results of the study are depicted below in Table 1. As can be seen, even at the highest dose (50 μg CT), powder injection to the skin did to show any signs of toxicity whereas 10 μg CT, when administered directly to the nasal mucosa resulted in 100% mortality. These results demonstrate that adjuvant toxicity can be avoided by administering vaccine compositions to skin via needleless powder injection.

TABLE 1

| | Intranasal | Powder Injection |
|---|---|---|
| CT Dose | #mortality/total | #mortality/total |
| 1 μg | 0/8 | 0/8 |
| 5 μg | 0/8 | 0/8 |
| 10 μg | 0/8 | 0/8 |
| 20 μg | 0/8 | 0/8 |
| 50 μg | 0/8 | 0/8 |

### Example 7

### Detection of Influenza-Specific IRA Secreting Cells in Mucosal Tissue After Skin Immunization

In order to quantify the number of antigen-specific IgA secreting cells present in mucosal tissue of mice immunized via powder injection to skin, the following study was carried out. The PR8 strain of inactivated influenza virus was used as the antigen of interest in this study. 5 μg of inactivated influenza virus (strain PRS/38, H1N1) was combined with 10 μg of the CT adjuvant and 10 μg of CpG DNA (CpG sequence: ATCGACTCTCGAGCGTTCTC (SEQ ID NO: 2)) and formulated as a particulate (powder) composition. The powdered vaccine composition was administered to an experimental group of 8 Balb/C mice using a powder injection device. Immunizations were carried out on days 0 and 28 of the study.

Two control groups were also established. For the control vaccines, 5 μg of inactivated influenza virus (strain PR8/38, H1N1) was combined with 10 μg of the CT adjuvant and 10 μg of CpG DNA (CpG sequence ATCGACTCTCGAGCGTTCTC (SEQ ID NO: 2)) and then mixed with phosphate buffered saline (PBS) to provide a liquid composition. The liquid vaccine composition was administered to a first control group of 8 Balb/C mice using a conventional needle and syringe to deliver the vaccine subcutaneously (SC). The liquid vaccine composition was also administered to a second control group of 8 Balb/C mice intranasaly. As with the experimental group, immunizations were carried out on days 0 and 28 of the study.

Two weeks after the second immunizations (i.e., day 42 of the study), mucosal tissue samples (trachea, small intestine, lung tissue and Peyer's patch) were collected from the mice in each group, and the samples were pooled separately for use in either a fragment culture assay to assess mucosal antibody production or in an ELISPOT assay to assess influenza-specific IgA secreting cells. More particularly, small pieces of trachea and small intestines were collected and washed extensively with calcium- and magnesium-free Hank's balanced salt solution (“HBSS,” GIBCO-BRL, Grand Island, N.Y.) containing 0.1% gentamicin. The tissue fragments were finally washed with complete RPMI media and cultured in 24-well flat-bottomed tissue culture plates (COSTAR) for 7 days under 90% O.sub.2 and 10% CO.sub.2 at 37.degree. C. The culture media was Kennett's H—Y medium (JRH BioSciences, Zlenexa, Kans.) containing 10% fetal calf serum, 1% L-glutamin, 0.01% gentamicin, and 1% antibiotic-antimycotic solution. Influenza-specific antibodies in the tissue culture supernatant were assayed using an ELISA (data not shown).

In the IgA ELISPOT the assays were performed using single cell suspensions prepared from the lungs and Peyer's patches of the immunized mice. To prepare the single cell suspensions, lungs were collected and digested with a Dipase solution (1.5 mg/ml in HBSS, Boehringer Mannheim Biochemicals, Indianapolis, Ind.) for 2 hours with continuous stirring. Peyer's patches were collected and teased apart using fine forceps. The fractionated mixtures were then passed through a 70 μm sterile cell strainer (Fisher Scientific, Pittsburgh, Pa.) to obtain single cell suspensions. The cells were then washed extensively with calcium- and magnesiom-free HBSS and then resuspended in a RPM medium containing 10% fetal calf serum, 1% L-glutamine, and 1% antibiotic-antimycotic solution.

The ELISPOT assays were carried out in 96-well nitrocellulose plates (Milliscreen, Millipore). The plates were coated with 10 μg/ml of formalin-inactivated PR8 influenza virus in 0.1M bicarbonate buffer (pH 9.5). Plates were blocked with 10% RPM for one hour at room temperature. After washing, 100 μl of lymphocyte suspensions from the mucosal tissues at varying concentrations were placed in each well, and the plated incubated overnight at 37.degree. C. in 5% CO.sub.2. The plates were then washed five times with PBS, and 100 μl of appropriately diluted goat anti-mouse IgA-alkaline phosphatase conjugate (Southern Biotechnologies) in PBS was added to each well, after which the plates were incubated at room temperature for 4 hours. The plates were then washed five times with PBS (0.05% Tween-80). The plates were then developed using an alkaline phosphatase substrate kit (Bio-Rad Laboratories, Melville, N.Y.). Spots were quantified by counting spots under an Olympus® dissecting microscope (Leeds Precision Instruments, Inc., Minneapolis, Minn.).

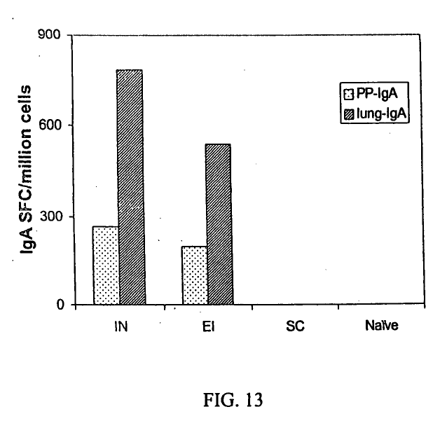

The results of the ELISPOT assay are depicted in FIG. 13. As can be seen, transdermal powder injection of mice using the PR8 vaccine composition adjuvanted with CT and CpG induced frequencies of IgA forming spots from Peyer's patch and lung mucosal tissue of 200 and 542 per million cells, respectively. The mice receiving the same vaccine composition directly to mucosal tissue (intranasal delivery) displayed only marginally higher frequencies, that is, 266 and 783 per million cells from the Peyer's patch and lung tissues samples, respectively. These results were in contrast to those seen with the mice receiving the same vaccine composition via subcutaneous injection, wherein influenza-specific IgA secreting cells were not detectable in the assay. These data convincingly demonstrate that administration of the powdered compositions of the present invention to skin induce a robust mucosal immune response substantially equivalent to that obtained with direct mucosal immunization techniques (intranasal).

Accordingly, novel vaccine compositions and methods of inducing a mucosal immune response are disclosed. Although preferred embodiments of the subject invention have been described in some detail, it is understood that obvious variations can be made without departing from the spirit and the scope of the invention as defined by the appended claims.

What is claimed is:

1. A method of generating a mucosal immune response at a mucosal surface, said method comprising delivering a particulate vaccine composition into or across the skin of a vertebrate subject using a transdermal delivery technique, wherein the vaccine composition comprises an antigen or a nucleic acid encoding said antigen.

2. The method of claim 1 wherein the particulate vaccine composition is delivered using a needleless syringe powder injection device.

3. The method of claim 1 wherein the mucosal immune response is specific for the antigen or is characterized by an IgA antibody response specific for the antigen.

4. The method of claim 1 wherein the antigen is derived or obtained from a pathogen that enters a subject's body via a mucosal surface or is selected from the group consisting of a viral antigen, a bacterial antigen or a live, attenuated organism.

5. The method of claim 1 further comprising the step of coadministering an adjuvant composition to the vertebrate subject.

6. The method of claim 5 wherein the adjuvant composition is particulate.

7. The method of claim 6 wherein the particulate adjuvant composition is delivered into or across the subject's skin using a transdermal delivery technique.

8. The method of claim 5 wherein the vaccine composition and the adjuvant composition are administered to the same site in the subject or are administered concurrently.

9. The method of claim 8 wherein the vaccine composition and the adjuvant composition are combined to provide a single composition.

10. The method of claim 9 wherein the vaccine composition is administered to the subject from a needleless syringe powder injection device.

11. The method of claim 5 wherein the mucosal immune response is specific for the antigen or is characterized by an IgA antibody response specific for the antigen.

12. The method of claim 5 wherein the antigen is derived or obtained from a pathogen that enters a subject's body via a mucosal surface.

13. The method of claim 5 wherein the adjuvant composition comprises an oligonucleotide containing a CpG motif and/or an ADP-ribosylating toxin.

14. The method of claim 13 wherein the adjuvant composition comprises a cholera toxin.

15. The method of claim 5 wherein the adjuvant composition comprises a combination of two or more adjuvants.

16. A particulate vaccine composition suitable for delivery into or across skin of a vertebrate subject, said composition comprising:
(1) an antigen or a nucleic acid encoding said antigen;
(2) an ADP-ribosylating toxin as an adjuvant; and
(3) an oligonucleotide containing a CpG motif.

17. The vaccine composition of claim 16 wherein the ADP-ribosylating toxin is a cholera toxin.

18. The vaccine composition of claim 16 wherein the antigen is derived or obtained from a pathogen that enters a subject's body via a mucosal surface or is selected from the group consisting of a viral antigen, a bacterial antigen or a live, attenuated organism.

19. A method for treating or preventing a disease caused by the entry of a pathogen into the body of a vertebrate subject via a mucosal surface, said method comprising:
(a) administering a particulate vaccine composition into or across skin of the subject, wherein the vaccine composition comprises an antigen derived or obtained from the pathogen, or a nucleic acid encoding said antigen; and
(b) coadministering an adjuvant composition to the subject, wherein the adjuvant composition comprises an ADP-ribosylating toxin and/or an oligonucleotide containing a CpG motif, and further wherein coadministration of the vaccine and adjuvant compositions is sufficient to bring about a mucosal immune response specific for the antigen.

20. The method of claim 19 wherein the ADP-ribosylating toxin is a cholera toxin.
